(19) United States
(12) Patent Application Publication
(10) Pub. No.: US 2022/0384039 A1
(43) Pub. Date: Dec. 1, 2022

(54) CANCER TYPE PREDICTION SYSTEM

(71) Applicant: GSI Technology Inc., Sunnyvale, CA (US)

(72) Inventor: Elona EREZ, Tel Aviv (IL)

(21) Appl. No.: 17/731,282

(22) Filed: Apr. 28, 2022

Related U.S. Application Data
(60) Provisional application No. 63/193,616, filed on May 27, 2021.
(60) Provisional application No. 63/330,323, filed on Apr. 13, 2022.

(51) Int. Cl. G16H 50/20; G16H 10/60; G06N 3/08

(52) U.S. Cl. CPC G16H 50/20; G16H 10/60; G06N 3/08

(57) ABSTRACT

A system for cancer type prediction includes a trained neural network (NN), a patient feature-set extractor (PFE), and an associative feature-set searcher (FSS). The trained NN receives a patient input vector from a patient record and generates cancer type predictions. The PFE extracts a known cancer feature set from patient input vector from a patient record with a known cancer type, and an unknown cancer feature set from a patient input vector from a patient record without a known cancer type, when passed through the trained NN. The FSS stores a known cancer feature set in a first portion of a column, and metadata in a second portion of a column, and finds K nearest neighbors of the unknown cancer feature set from among the stored known cancer feature sets.

### CROSS REFERENCE

This application claims priority from U.S. provisional patent applications 63/193,616, filed May 27, 2021, and 63/330,323, filed Apr. 13, 2022, both of which are incorporated herein by reference.

### FIELD OF THE INVENTION

The present invention relates to cancer prediction generally and to methods of prediction using neural networks in particular.

### BACKGROUND OF THE INVENTION

For healthcare professionals and researchers, the major focus of cancer prediction is on cancer susceptibility, recurrence, and prognosis. Precise prediction of cancer types is vital for cancer diagnosis and therapy. Important cancer marker genes can be inferred through predictive modelling, for example using neural networks. Cancer detection is the classification of tumor types and identification of markers for each cancer.

One example takes unstructured gene expression inputs to a convolutional neural network (CNN) model that classifies tumor and non-tumor samples into their designated cancer types or as normal. This is described in the paper “Convolutional neural network models for cancer type prediction based on gene expression” by Milad Mostavi, Yu-Chiao Chiu, Yufei Huang and Yidong Chen, Apr. 3, 2020.

Another example takes somatic genomic alterations (SGAs) inputs to a neural network configured as a genomic impact transformer (GIT) which predicts differentially expressed genes (DEGs) in tumors. This is described in the published paper “From genome to phenome: Predicting multiple cancer phenotypes based on somatic genomic alterations via the genomic impact transformer” by Yifeng Tao, Chunhui Cai, William W. Cohen, and Xinghua Lu, presented at the Pacific Symposium on Biocomputing 2020.

### SUMMARY OF THE PRESENT INVENTION

There is therefore provided, in accordance with a preferred embodiment of the present invention, a method for cancer type prediction. The method includes training a neural network (NN) to receive a patient input vector from a patient record and to generate cancer type predictions, extracting a known cancer feature set from the patient input vector from a patient record with a known cancer type passed through the NN, and storing the known cancer feature set in a first portion of a column and the known cancer type in a second portion of a column of an associative feature-set searcher (FSS). The method also includes second extracting an unknown cancer feature set from the patient input vector, from a patient record without a known cancer type passed through the NN, finding K nearest neighbors of the unknown cancer feature set from among stored known cancer feature sets using the FSS, and providing the cancer type prediction associated with the K nearest neighbors of the unknown cancer feature set, together with data from the patient records associated with the K nearest neighbors of the unknown cancer feature set.

Moreover, in accordance with a preferred embodiment of the present invention, the NN is a convolutional neural network (CNN) or a genomic impact transformer (GIT).

Further, in accordance with a preferred embodiment of the present invention, the patient input vector is an unstructured gene expression vector or a somatic genomic alteration.

Still further, in accordance with a preferred embodiment of the present invention, the patient record contains patient details, known cancer type, prognosis, medication, or therapy.

Additionally, in accordance with a preferred embodiment of the present invention, the metadata contains a patient record identifier, the known cancer type, or the patient record.

There is therefore provided, in accordance with a preferred embodiment of the present invention, a system for cancer type prediction. The system includes a trained neural network (NN), a patient feature-set extractor (PFE), an associative feature-set searcher (FSS), and an output coordinator. The trained NN receives a patient input vector from a patient record and generates cancer type predictions. The PFE extracts a known cancer feature set from patient input vector from a patient record with a known cancer type, and an unknown cancer feature set from a patient input vector from a patient record without a known cancer type, when passed through the trained NN. The FSS stores a known cancer feature set in a first portion of a column, and metadata in a second portion of a column, and finds K nearest neighbors of the unknown cancer feature set from among the stored known cancer feature sets. The output coordinator provides the cancer type prediction associated with the K nearest neighbors of the unknown cancer feature set, together with patient data from the patient records associated with the K nearest neighbors of the unknown cancer feature set.

Moreover, in accordance with a preferred embodiment of the present invention, the trained NN is a convolutional neural network (CNN) or a genomic impact transformer (GIT).

Further, in accordance with a preferred embodiment of the present invention, the patient input vector is an unstructured gene expression vector or a somatic genomic alteration.

Still further, in accordance with a preferred embodiment of the present invention, the patient record contains patient details, known cancer type, prognosis, medication, or therapy.

Additionally, in accordance with a preferred embodiment of the present invention, the metadata contains a patient record identifier, the known cancer type, or the patient record.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a schematic illustration of an NN-KNN cancer prediction system, constructed and operative in accordance with a preferred embodiment of the present invention

FIGS. 2A and 2B are schematic illustrations of a neural network comprising multiple neural layers, nodes and connections;

FIG. 3 is a schematic illustration of an exemplary similarity search system implemented in associative memory array;

FIGS. 4A and 4B are schematic illustrations of a first embodiment of a neural network (NN) useful in the system of FIG. 3, under training and during inference, respectively; and

FIGS. 5A and 5B are schematic illustrations of a second embodiment of a neural network (NN) useful in the system of FIG. 3, under training and during inference, respectively.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

Applicant has realized that, by integrating K-Nearest Neighbor (KNN) search techniques with a neural network (NN) for cancer type prediction, patient data of the K nearest neighbor candidate patients may also be made available as part of the system output. It will be appreciated that such patient data may enable healthcare professionals to leverage other patient data and treatment regimens along with a cancer diagnosis.

Applicant has also realized that such an NN-KNN cancer prediction system may improve the accuracy of cancer predictions.

### NN-KNN Cancer Type Prediction System

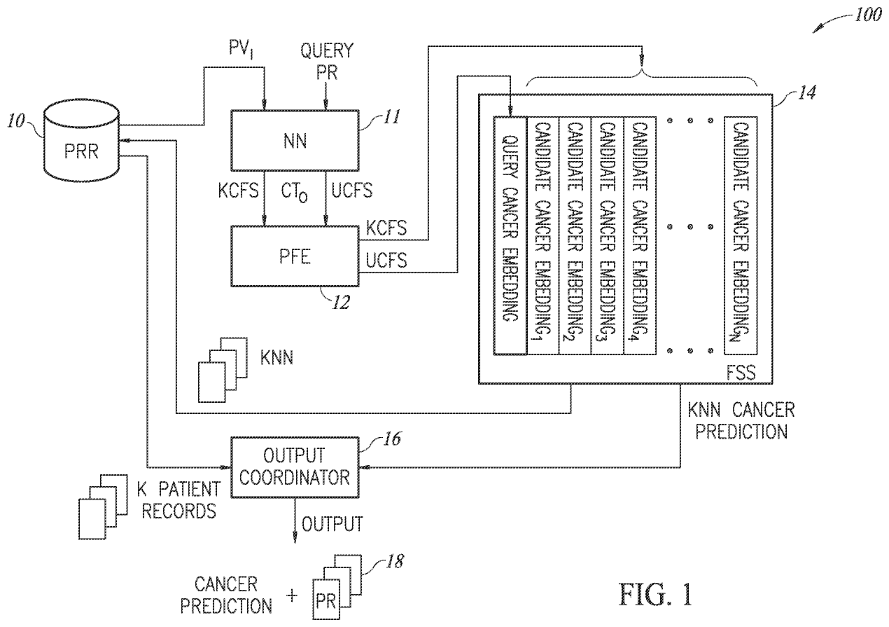

Reference is made to FIG. 1 which is an illustration of an NN-KNN Cancer Prediction System 100. The system comprises a patient record repository (PRR) 10, a neural network (NN) 11, a patient feature-set extractor (PFE) 12, an associative feature-set searcher (FSS) 14, and an output coordinator 16. PRR 10 may contain plurality of patient records 18 including a required input patient vector PVi, such as an SGA vector, and cancer type output CTo, along with any other medical or therapeutic information related to the patient.

NN 11 may be cancer trained as described herein below, using all, some or none of patient records 18. If patient records 18 are not used to train NN 11, a special training record set may be used which may be stored in PRR 10 or elsewhere (not shown).

Researchers utilize neural networks, which are mathematical models, to recognize properties of input data. These may be implemented on software platforms such as Rdkit, Deepchem and others. A neural network is made up of nodes that are arranged into layers, and connected to other nodes.

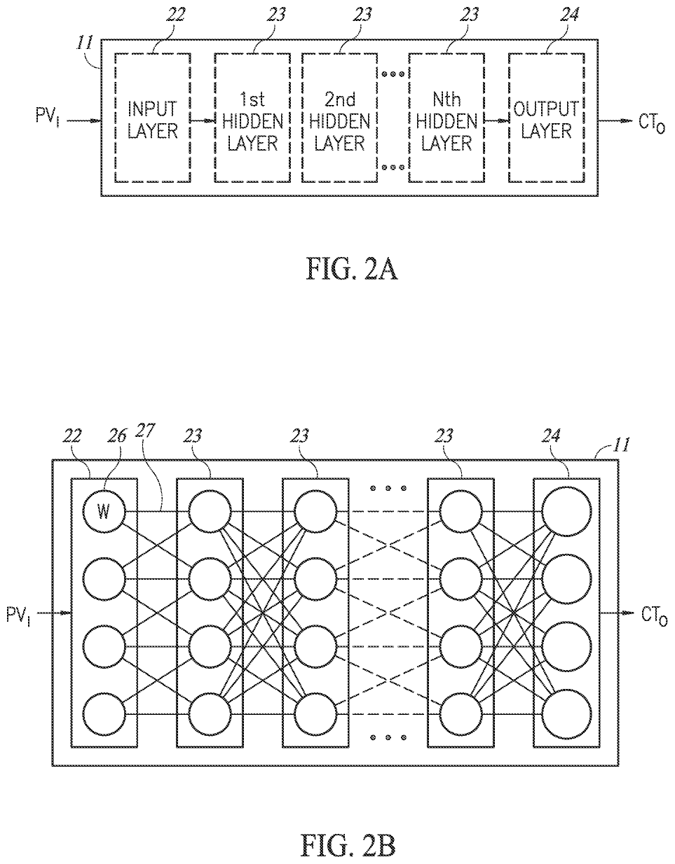

Reference is now made to FIGS. 2A and 2B, which illustrate NN 11 comprising multiple neural layers; an input layer 22, a plurality of hidden layers 23, and an output layer 24. Each layer comprises a plurality of nodes 26, and the nodes in each layer may be connected by a plurality of connections 27. Each node may be fully connected to each node in the previous and subsequent layers, but is not required to be as such.

Input vector PVᵢ, which, in this application may represent the structure and features of a DNA sample, as described in detail hereinbelow, enters NN 11 at input layer 22, traverses hidden layers 23 and exits NN 11 as an output vector CTₒ at output layer 24.

There are two main modes of operating NN 11: a training mode and an inference mode (which includes testing, verification and regular use of NN 11). During training, input vector PVᵢ, with a known output value of CTₒ, is put through NN 11. The nodes 26, weights W, connections 27 and other features of NN 11 (shown in FIG. 2B) are adjusted, for example by cross entropy loss, so when PVᵢ traverses NN 11, NN 11 transforms PVᵢ to the known value of CTₒ at output layer 24. Training an NN to perform accurate transformations is a complex task, as is known in the art. An NN 11 under training may contain special layers 23, specifically to aid in training the network, which when training is complete may be removed or disconnected.

Once NN 11 is trained, another set of input vectors is used to test and verify if the network transformation is reliable and accurate. Another set of test input vectors, again with known output values, is passed through NN 1 and actual CTₒ results are compared against known CTₒ values. If the results are acceptable, then NN 11 is considered trained. Once trained, NN 11 may be used in ‘inference’ mode to predict the output of unknown query vectors.

Researchers strive to create the perfect transformation model within an NN that will generate a desired output for a given input. For example, DNA aberration or gene expression properties, called features, may be input to an NN, and the cancer properties of such inputs may be predicted at the output. As known by those in the art, during the training phase of an NN, various deep learning techniques are used to refine the NN. These techniques include, but are not limited to neighbor feature aggregation layers, normalization layers, pooling layers, non-linear transformation layers, readout layers, and others. Current NN techniques are described in the website publications, Deep Learning, at http://www.deeplearningbook.org; in the article “SimGNN: A Neural Network Approach to Fast Graph Similarity Computation” published by ACM 2019; and, ‘Semi-Supervised Classification with Graph Convolutional Networks’ published by ICLR 2017, all of which are incorporated herein by reference.

### ‘Feature Sets’ or ‘Embeddings’

In accordance with a preferred embodiment of the present invention, unlike during training, during inference NN 11 may not be utilized to output a cancer prediction from output layer 24 at all. Instead, NN 11 may utilize only hidden layers 23 and PFE 12 may extract feature sets from within the final layers of NN 11.

Applicant has realized that in a cancer trained NN, as the input vector, comprising a plurality of cancer features, traverses from the input layer and through a plurality of hidden layers, its cancer features may be transformed, for example, to tumor feature sets, sometimes called ‘tumor embeddings,’ before being further transformed into the cancer type property at the output layer. Applicant has also realized that, rather than use the cancer type property from such a cancer trained NN, tumor feature sets may be extracted from within the hidden layers of the NN and used outside of the NN to mathematically compare their cancer properties with other extracted tumor embedding vectors. These feature sets may be compared using a KNN type search system such as FSS 14.

Such feature sets may be a known cancer feature set if the cancer type associated with the feature set is known, or an unknown cancer feature set if the cancer type associated with the feature set is not known. For example, PFS 12 may extract a vector of 128 features and may store it in columns of FSS 14, which may be implemented on an associative processing unit as described hereinbelow.

### Associative Processing Units and KNN Search

Associative memory computational units called associative processing units (APUs), such as the Gemini APU, commercially available from GSI Technologies Inc. of the USA, may store multibit data in parallel columns. and may operate on all similar bits of such multibit data, stored in rows, simultaneously with a constant processing time, regardless of the number of columns. APUs can be utilized to perform simple Boolean logic functions and complex search functions on data in columns, as described in U.S. Pat. No. 8,238,173 (entitled “USING STORAGE CELLS TO PERFORM COMPUTATION”) dated Aug. 7, 2012; U.S. Pat. No. 9,859,005 (entitled “MEMORY DEVICE”) dated Jan. 2, 2018; U.S. Pat. No. 10,153,042 (entitled “IN-MEMORY COMPUTATIONAL DEVICE WITH BIT LINE PROCESSORS”) dated Dec. 11, 2018, all assigned to Applicant and all incorporated herein by reference.

Applicant has realized that a known cancer feature set may be extracted from a neural network and may be loaded into columns of FSS 14, to be used as a database of known cancer embeddings. Such known cancer feature sets stored in a database may be compared in parallel to an unknown cancer feature set using, for example, a KNN search. A predesignated number of candidates, K, may then be selected and then all K cancer types from the K candidates may be returned to the medical professional to use in their diagnosis. If the value of K is only 1, then that may be returned as the prediction.

One exemplary similarity search is described in U.S. Pat. No. 10,929,751, entitled “Finding K Extreme Values in Constant Processing Time,” dated Feb. 23, 2021, and US patent publication 2021/0287762, entitled “Molecular Similarity Search,” published on Sep. 16, 2021, which are both commonly owned by the Applicant of the present invention and are both incorporated herein by reference.

In a KNN similarity search, which is well known in the art, the similarity between each known cancer feature set and an unknown cancer feature set may be computed using Euclidian or Hamming distance metrics between each of the features and then a final score may be computed, for example, as the sum of such distances.

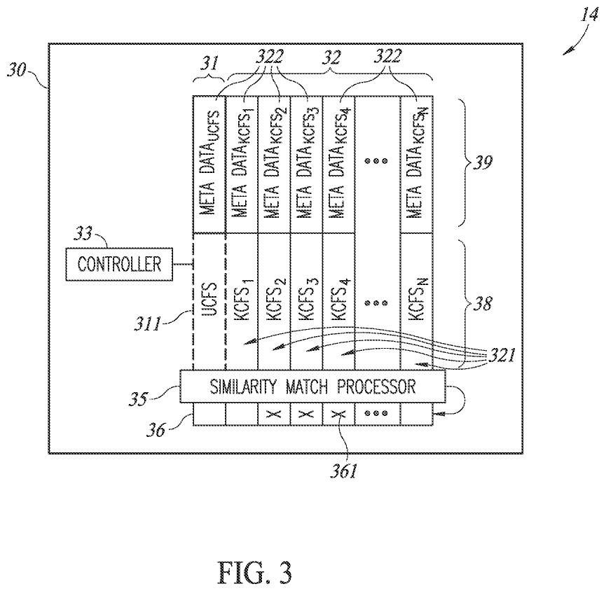

Reference is now made to FIG. 3 which illustrates an exemplary FSS 14, implemented in associative memory array 30. FSS 14 comprises a query data column 31, a large plurality of candidate data columns 32, a controller 33, a similarity match processor 35, and a match row 36. Data columns 31 and 32 may comprise a first portion 38 and a second portion 39. Match row 36 comprises a per-column match bit 361.

Multiple known cancer feature sets (321) may be loaded, as described hereinabove, into a first portion 38 of memory columns 32. Column 31 and columns 32 may comprise a second portion 39 that may contain metadata (322) attached to the known cancer feature sets (321) or unknown cancer feature set (311) such as a link to a patient record 18 in PRR 10, but such a second portion 39 may not be used as part of the KNN search itself. Unknown cancer feature set (311) may be loaded into a first portion 38 of query data column 31. Similarity match processor 35 may perform a similarity search in parallel between unknown cancer feature set (311) in column 31 and the large plurality of known cancer feature sets (321) in columns 32, generating per-column match results containing bit indications (361) of which columns 32 were similarity matched to column 31 and which were not similarity matched. Similarity match processor 35 may write these bit indication (361) results into match row 36 in the lower section of array 30.

It will be appreciated that the number of matched columns may equal the number of K nearest neighbors required by the KNN search.

A plurality of PRs 38 with a known cancer diagnosis may be passed through NN 31, and their known cancer feature sets (321) extracted by PFE 12 and stored in FSS 14 as candidate vectors. Then a patient record 18 of unknown cancer type may be passed through NN 11, and the unknown cancer feature set (311) extracted by PFE 12 and stored in FSS 14 as a query vector. As discussed herein above, FSS 14 may perform a KNN search and may mark the columns 32 containing the K most similar known cancer feature sets (321).

As mentioned hereinabove, the known cancer type may be stored as a second part 39 of a candidate vector. When FSS 14 has identified the K nearest neighbors to the unknown cancer feature set (311), it may then output the cancer type as described hereinabove.

It will be appreciated that due to the parallel processing within an associative processing system, searches may be performed in a fixed time, unaffected by the number of candidate vectors. It will also be appreciated that NN-KNN cancer type prediction system 100 may deliver more accurate cancer prediction, in a shorter processing time similar to other feature-set based KNN systems.

### Leveraging Additional Patient Data

As mentioned hereinabove each candidate vector may also contain, in the second portion 39 of column 31 and columns 32, a link to the patient record 18 from which it was extracted. In another embodiment, patient data from patient record 18 itself may be stored as part of second portion 39. and returned as part of a KNN search directly by FSS 14.

It will also be appreciated that, unlike other NN cancer prediction systems, NN-KNN cancer type prediction system 100 may not require further training when additional patient records are added. Instead, new records may be run through NN 11 and known cancer feature sets (321) may be extracted by PFE 12 and stored as further candidates in FSS 14.

It will also be appreciated that in another embodiment, the input vector PVi and the NN utilized may be varied to produce cancer prediction from different types of input vectors. This may enable healthcare professionals to leverage a multiplicity of parallel patient data sources, to not only more accurately predict cancer type, but to gain knowledge of from multiple parallel patient datasets.

Cancer Type Prediction from Gene Expressions Using a CNN

Applicant has realized that system 100 may be utilized to predict cancer type from gene expressions as discussed in the published paper “Convolutional neural network models for cancer type prediction based on gene expression” by Milad Mostavi, Yu-Chiao Chiu, Yufei Huang, Yidong Chen, Apr. 3, 2020.

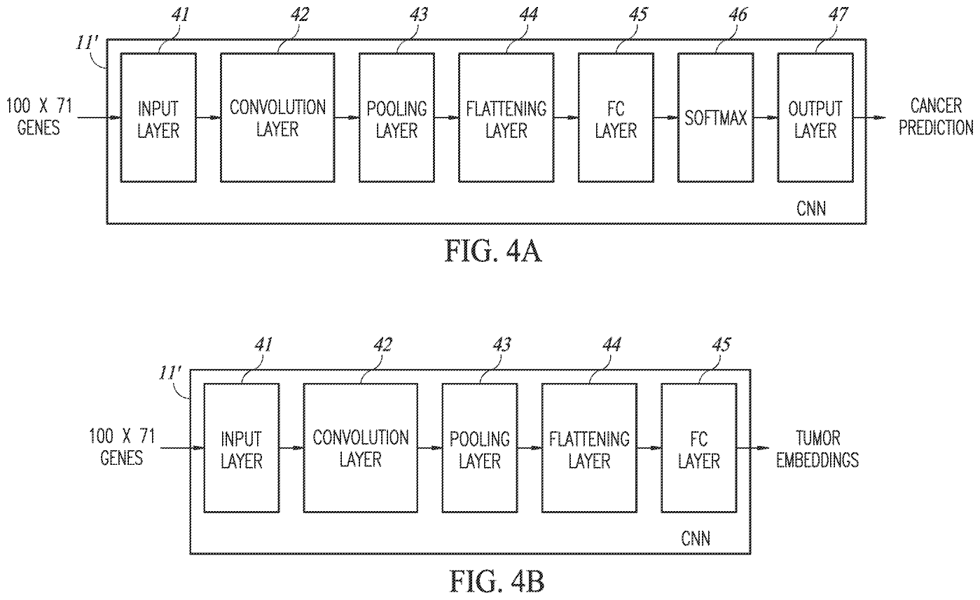

In this preferred embodiment, and as shown in FIGS. 4A and 4B, unstructured gene expression vectors may be input to NN 11 (as shown in FIG. 1) which is configured as a cancer trained CNN 11′, which predicts tumor and non-tumor classifications. The design of CNN 11′ takes into consideration the effects of tissue of origin that can potentially bias the identification of cancer markers. The design of CNN 11′ is constrained to include only one layer of convolution, as shallower models are preferred for problems such as cancer type prediction, where there are limited samples relative to the number of parameters. Such shallow models avoid overfitting and also demand fewer resources for training.

In this embodiment unstructured gene expression vectors may be input into CNN 11′ and a cancer type prediction output. During training, unstructured gene expression vectors with a known cancer type are input into CNN 11′ and CNN 11′ trained, as described hereinabove, to output the correct cancer type. FIG. 4A illustrates CNN 11′ configured for training, comprising seven layers: an input layer 41, a convolution layer 42, a pooling layer 43, a flattening layer 44, an FC layer 45, a SoftMax layer 46 and an output layer 47.

Training data used for this embodiment was downloaded from the pan-cancer RNA-Seq data containing 10340 samples for 33 cancer types and 731 samples for 23 normal tissues, was downloaded from The Cancer Genome Atlas (TCGA) project. Gene expression was represented by log 2(FPKM+1), where FPKM is the number of fragments per kilobase per million mapped reads. Genes with low information burden (mean <0.5 or st. dev. <0.8) across all TCGA samples, regardless of their cancer types, were removed. From the complete downloaded dataset of genes, a selection of 7,091 genes with relatively higher overall expression and high variability was chosen, in order to reduce the number of non-informative, or noise-sensitive features. In order to round input dimensions and facilitate modeling, nine zeros were added to the gene expressions in order to fix the vector length at 7,100 bits.

Once trained, CNN 11′ may be reconfigured for inference mode. Traditionally, a trained NN when used in inference mode, may be used as a predictor for inputs with unknown output types. In this embodiment, unstructured gene expression vectors could have been input to CNN 11′ in inference mode and used to predict a cancer type on the output. However, as mentioned hereinabove, cancer type feature sets may be extracted from within the layers of CNN 11′. In this embodiment, cancer feature sets, each of 128 features per patient, may be extracted from FC layer 45, as illustrated in FIG. 4B. FIG. 4B illustrates CNN 11′ configured for inference, comprising only five layers: input layer 41, convolution layer 42, pooling layer 43, flattening layer 44, and FC layer 45.

Among 34 classes (33 cancers and 1 normal), when CNN 11′ was used as a regular cancer predictor in inference mode as defined by Mostavi et Al in their paper, then the prediction accuracy was 94.5%. However, when CNN 11′ was configured as for this embodiment as described hereinabove, and utilized as part of NN-KNN system 100 as described hereinabove, then prediction accuracy increased to 95.8%.

Applicant has realized that future improvements may further improve accuracy. Such improvements may include training a new linear neural network for dimension reduction, and increasing accuracy using longer feature set vectors of 512 or 1024 features, rather than the 128 described hereinabove.

Cancer Type Prediction from SGAs Using a GIT

Applicant has also realized that system 100 may be utilized to predict cancer type from somatic genomic alterations (SGAs) as discussed in the published paper “From genome to phenome: Predicting multiple cancer phenotypes based on somatic genomic alterations via the genomic impact transformer” by Yifeng Tao, Chunhui Cai, William W. Cohen, and Xinghua Lu, presented at the Pacific Symposium on Biocomputing 2020. The paper discusses how cancers are mainly caused by somatic genomic alterations (SGAs) that perturb cellular signaling systems and eventually activate oncogenic processes. Tao et Al describe an NN configured as a GIT. A GIT is a deep neural network model with encoder-decoder architecture, that infers the functional impact of SGAs on cellular signaling systems, by modeling the statistical relationships between SGA events and differentially expressed genes (DEGs) in tumors. In their paper, Tao et Al trained their GIT using SGAs, and known cancer types associated with those SGAs as input, and adjusted the GIT to output the known DEGs associated with the SGA inputs. As such Tao et Al's GIT was trained to predict DEG outputs given SGA and known cancer type inputs.

Applicant has realized that by changing the GIT training such that cancer type was switched to become an output, Tao et Al's GIT could be trained as a cancer type predictor.

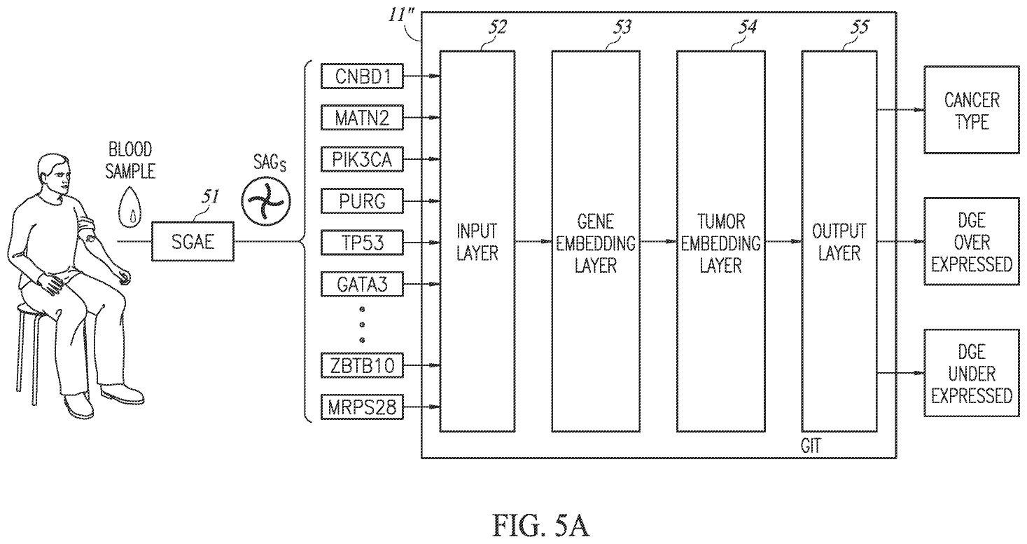

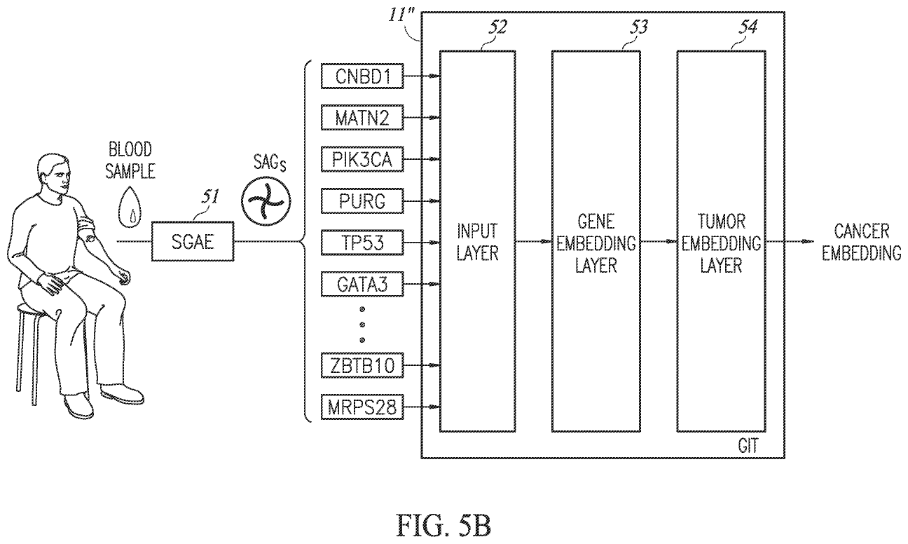

In this preferred embodiment, and as shown in FIGS. 5A and 5B, SGAs may be input into NN 11 (as shown in FIG. 1) which is configured as a GIT 11″.

In this embodiment SGA vectors may be input into GIT 11″ and a cancer type prediction output, along with DEG predictions. During training, SGA vectors with a known cancer type are input into GIT 11″ and CNN 11′ trained, as described hereinabove, to output the correct cancer type. FIG. 5A illustrates GIT 11″, configured for training, comprising and SGA Extractor (SGAE) 51 (to extract SGAs from a patient blood sample), an input layer 52, a gene embedding layer 53, a tumor embedding layer 54, and an output layer 55. Training data of 4,468 profiled tumors used for this embodiment was downloaded from The Cancer Genome Atlas (TCGA) project.

Once trained, GIT 11″ may be reconfigured for inference mode. Also in this embodiment, SGA vectors could have been input to GIT 11″ in inference mode and used to predict a cancer type on the output. However, as mentioned hereinabove, cancer type feature sets may be extracted from within the layers of GIT 11″. FIG. 5B illustrates GIT 11″ configured for inference, comprising only three layers: input layer 52, gene embedding layer 53, and tumor embedding layer 54.

When configured to output a cancer type prediction in inference mode, similar to GIT 11″ shown in FIG. 5A), the prediction accuracy of GIT 11″ was 55%. However, when GIT 11″ was configured as for this embodiment as described hereinabove, and utilized as part of NN-KNN system 100 as described hereinabove, then prediction accuracy increased to 59%.

What is claimed is:

1. A method for cancer type prediction, the method comprising:
training a neural network (NN) to receive a patient input vector from a patient record and to generate cancer type predictions;
extracting a known cancer feature set from said patient input vector from a patient record with a known cancer type passed through said NN;
storing said known cancer feature set in a first portion of a column, and said known cancer type in a second portion of a column of an associative feature-set searcher (FSS);
second extracting an unknown cancer feature set from said patient input vector, from a patient record without a known cancer type passed through said NN;
finding at least one K nearest neighbors of said unknown cancer feature set from among at least one stored said known cancer feature set using said FSS; and
providing said cancer type prediction associated with said at least one K nearest neighbors of said unknown cancer feature set, together with data from said patient records associated with said at least one K nearest neighbors of said unknown cancer feature set.

2. The method of claim 1 wherein said NN is one of a convolutional neural network (CNN) and a genomic impact transformer (GIT).

3. The method of claim 1 wherein said patient input vector is one of an unstructured gene expression vector and a somatic genomic alteration.

4. The method of claim 1 wherein said patient record contains at least one of patient details, known cancer type, prognosis, medication, and therapy.

5. The method of claim 1 wherein said metadata contains at least one of a patient record identifier, said known cancer type, and said patient record.

6. A system for cancer type prediction, the system comprising:
a trained neural network (NN) to receive a patient input vector from a patient record and to generate cancer type predictions;
a patient feature-set extractor (PFE) to extract a known cancer feature set from patient input vector from a patient record with a known cancer type, and an unknown cancer feature set from a patient input vector from a patient record without a known cancer type, when passed through said trained NN;
an associative feature-set searcher (FSS) to store at least one said known cancer feature set in a first portion of a column, and metadata in a second portion of a column, and to find at least one K nearest neighbors of said unknown cancer feature set from among at least one stored said known cancer feature set; and
an output coordinator to provide said cancer type prediction associated with said at least one K nearest neighbors of said unknown cancer feature set, together with patient data from said patient records associated with said at least one K nearest neighbors of said unknown cancer feature set.

7. The system of claim 1 wherein said trained NN is one of a convolutional neural network (CNN) and a genomic impact transformer (GIT).

8. The system of claim 1 wherein said patient input vector is one of an unstructured gene expression vector and a somatic genomic alteration.

9. The system of claim 1 wherein said patient record contains at least one of patient details, known cancer type, prognosis, medication, and therapy.

10. The system of claim 1 wherein said metadata contains at least one of a patient record identifier, said known cancer type, and said patient record.
